(19) United States
(12) Patent Application Publication
(10) Pub. No.: US 2020/0107728 A1
(43) Pub. Date: Apr. 9, 2020

(54) INTEGRATED SENSOR AND HEALTH MONITOR

(71) Applicant: Wai Cho Ho, Shanghai City (CN)

(72) Inventor: Wai Cho HO, Shanghai City (CN)

(21) Appl. No.: 16/580,410

(22) Filed: Sep. 24, 2019

(30) Foreign Application Priority Data
Oct. 9, 2018 (CN) 201821638826.0

(51) Int. Cl. A61B 5/0205; A61B 5/022; A61B 5/00

(52) U.S. Cl. CPC A61B 5/0205; A61B 5/024; A61B 5/7425; A61B 5/02233

(57) ABSTRACT

Disclosed is an integrated sensor, including a first electrocardio-electrode, a second electrocardio-electrode and an oxygen saturation sensor, where an insulating member is disposed between the first electrocardio-electrode and the second electrocardio-electrode, and the first electrocardio-electrode is disposed at a side of the oxygen saturation sensor. Also disclosed is a health monitor, including a blood pressure bandage for measuring blood pressure and the aforementioned integrated sensor, where the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

### TECHNICAL FIELD

The present disclosure relates to the technical field of medical equipment, and in particularly, to an integrated sensor, and further to a health monitor.

### BACKGROUND

Existing integrated monitors mostly use different structures to measure oxygen saturation and electrocardiograms. For example, oxygen saturation is measured by means of a transmissive sensor in a finger-clamping mode, and an electrocardiogram is measured in a mode of connecting wires and electrode slices to various parts of the body. The structure of the measurement is complicated, and when it is required to measure a plurality of items together, the operation is troublesome.

### SUMMARY

In order to overcome the foregoing deficiencies of the prior art, the present disclosure provides an integrated sensor and a health monitor. The health monitor uses an integrated sensor, and the health monitor can measure a plurality of items simultaneously, and has a simple structure and is simple and convenient to operate.

The technical solution adopted by the present disclosure to solve the technical problems thereof is as follows:

An integrated sensor, including a first electrocardio-electrode, a second electrocardio-electrode and an oxygen saturation sensor, where an insulating member is disposed between the first electrocardio-electrode and the second electrocardio-electrode, and the first electrocardio-electrode is disposed at a side of the oxygen saturation sensor.

As an improvement of the foregoing technical solution, the side of the oxygen saturation sensor is surrounded by the first electrocardio-electrode.

As an improvement of the foregoing technical solution, a groove in which a finger can be placed is provided at an upper end of the first electrocardio-electrode.

As an improvement of the foregoing technical solution, the oxygen saturation sensor is disposed at a bottom of the groove.

As an improvement of the foregoing technical solution, the oxygen saturation sensor is a reflective sensor or a transmissive sensor.

A health monitor, including a blood pressure bandage for measuring blood pressure and the aforementioned integrated sensor, where the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

As an improvement of the foregoing technical solution, the health monitor further includes a host, where the host is connected to the integrated sensor by means of a wire, and the host is connected to the blood pressure bandage by means of the wire and an air tube.

As an improvement of the foregoing technical solution, the host is provided with a switch and a display screen, and information such as blood pressure, electrocardiogram, heart rate and oxygen saturation is displayed on the display screen.

The beneficial effects of the present disclosure are as follows:

The health monitor combines an integrated sensor with a blood pressure bandage. When the blood pressure bandage is tied, a second electrocardio-electrode is in contact with the skin of the arm or wrist at one side of a user, and the blood pressure, electrocardiogram, heart rate and oxygen saturation can be measured simultaneously only by pressing a first electrocardio-electrode and an oxygen saturation sensor with the finger of the other hand. The health monitor is simple in structure, saves the time for complicated operations, is convenient to use, facilitates people's health check and monitoring, and improves inspection efficiency.

### BRIEF DESCRIPTION OF THE DRAWINGS

The present disclosure will be further described below in conjunction with the accompanying drawings and specific embodiments. In the drawings,

FIGS. 1a-1b are schematic structural diagrams of an integrated sensor according to an embodiment of the present disclosure; and

FIGS. 2a-2b are a schematic structural diagrams of a health monitor according to an embodiment of the present disclosure.

### DETAILED DESCRIPTION

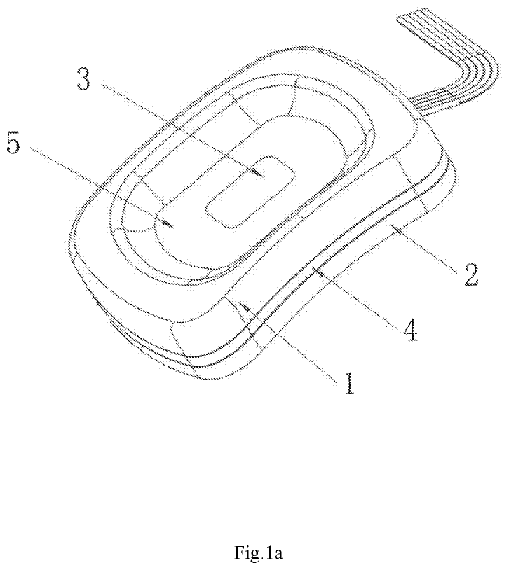

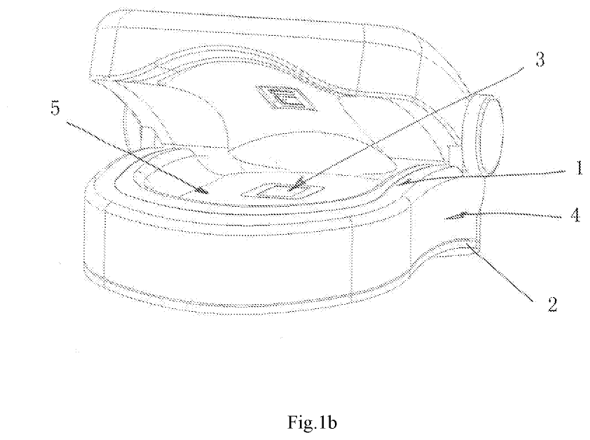

Referring to FIGS. 1a-1b, an integrated sensor provided by the present disclosure includes a first electrocardio-electrode 1, a second electrocardio-electrode 2, and an oxygen saturation sensor 3. An insulating member 4 is disposed between the first electrocardio-electrode 1 and the second electrocardio-electrode 2, and the first electrocardio-electrode 1 is disposed on a side of the oxygen saturation sensor 3, so that the finger can be simultaneously in contact with the first electrocardio-electrode 1 and the oxygen saturation sensor 3.

In this embodiment, the side of the oxygen saturation sensor 3 is surrounded by the first electrocardio-electrode 1. Specifically, a groove 5 in which a finger can be placed is provided at an upper end of the first electrocardio-electrode 1. The groove 5 facilitates a user to determine the position where the finger is placed, and the oxygen saturation sensor 3 is disposed at the bottom of the groove 5, to ensure that the finger can be in contact with the first electrocardio-electrode 1 when being in contact with the oxygen saturation sensor 3. The oxygen saturation sensor 3 is a reflective sensor.

Certainly, the oxygen saturation sensor 3 may also be a transmissive sensor. In this case, the integrated sensor is provided with a mounting piece above the groove 5. A transmitting device of the transmissive sensor is mounted at one of the lower end face of the mounting piece and the bottom of the groove, and a receiving device of the transmissive sensor is mounted at the other of the lower end face of the mounting piece and the bottom of the groove. During use, the finger is placed in the groove 5, and the transmitting device and the receiving device are located on both sides of the finger, respectively.

Moreover, a chip is further disposed in the integrated sensor, and the chip is electrically connected to the first electrocardio-electrode 1, the second electrocardio-electrode 2, and the oxygen saturation sensor 3. The chip processes signals sent out by the first electrocardio-electrode 1, the second electrocardio-electrode 2 and the oxygen saturation sensor 3, and a wire for transmitting signals output by the chip is provided at one side of the integrated sensor.

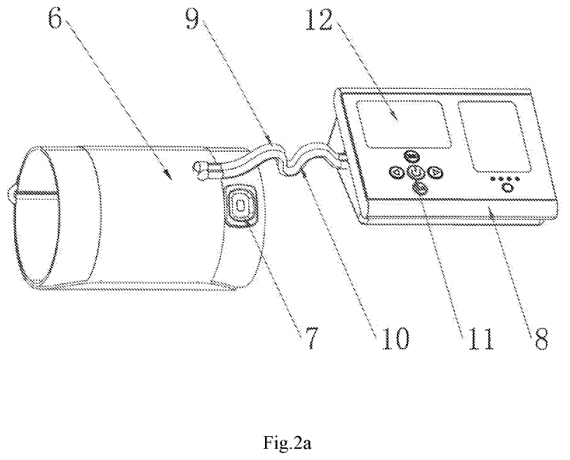

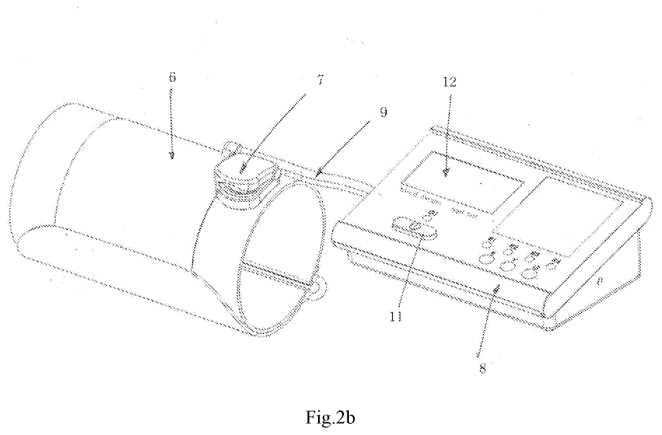

Further, referring to FIGS. 2a-2b, a health monitor includes a blood pressure bandage 6 for measuring blood pressure and an integrated sensor 7, where the integrated sensor 7 is disposed on a side of the blood pressure bandage 6 in a penetrating mode, the first electrocardio-electrode 1 faces towards the outside of the blood pressure bandage 6, and the second electrocardio-electrode 2 faces towards the inside of the blood pressure bandage 6. It is ensured that when the blood pressure bandage 6 is tied, the second electrocardio-electrode 2 can be in contact with the skin of the arm or wrist of a user.

Further, the health monitor further includes a host 8, where the host 8 is connected to the integrated sensor 7 by means of a wire 9, and the host 8 is connected to the blood pressure bandage 6 by means of the wire 9 and an air tube 10. The host 8 is provided with a switch 11 and a display screen 12, and information such as blood pressure, electrocardiogram, heart rate and oxygen saturation is displayed on the display screen 12.

During use, the blood pressure bandage 6 is tied to the arm or the wrist at one side of the user, the second electrocardio-electrode 2 is in contact with the skin of the arm or the wrist, and the blood pressure, electrocardiogram, heart rate and oxygen saturation are measured simultaneously by pressing the inside of the groove 5 with the finger of the other hand and turning on the switch. Information such as the blood pressure, electrocardiogram, heart rate and oxygen saturation is displayed by means of the display screen 12. The health monitor is simple in structure, saves the time for complicated operations, is convenient to use, facilitates people's health check and monitoring, and improves inspection efficiency.

The above descriptions are only preferred embodiments of the present disclosure, but the present disclosure is not limited to the above embodiments, as long as the technical effects of the present disclosure are achieved by any identical or similar means, the means should fall within the scope of protection of the present disclosure.

1. An integrated sensor, comprising:
a first electrocardio-electrode;
a second electrocardio-electrode;
an oxygen saturation sensor; and
an insulating member disposed between the first electrocardio-electrode and the second electrocardio-electrode, wherein the first electrocardio-electrode is disposed at a side of the oxygen saturation sensor.

2. The integrated sensor according to claim 1, wherein the side of the oxygen saturation sensor is surrounded by the first electrocardio-electrode.

3. The integrated sensor according to claim 1, wherein a groove in which a finger can be placed is disposed at an upper end of the first electrocardio-electrode.

4. The integrated sensor according to claim 2, wherein a groove in which a finger can be placed is disposed at an upper end of the first electrocardio-electrode.

5. The integrated sensor according to claim 3, wherein the oxygen saturation sensor is disposed at a bottom of the groove.

6. The integrated sensor according to claim 4, wherein the oxygen saturation sensor is disposed at a bottom of the groove.

7. The integrated sensor according to claim 1, wherein the oxygen saturation sensor is a reflective sensor or a transmissive sensor.

8. A health monitor, comprising:
a blood pressure bandage for measuring blood pressure;
and the integrated sensor according to claim 1;
wherein the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

9. A health monitor, comprising:
a blood pressure bandage for measuring blood pressure;
and the integrated sensor according to claim 2;
wherein the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

10. A health monitor, comprising:
a blood pressure bandage for measuring blood pressure;
and the integrated sensor according to claim 3;
wherein the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

11. A health monitor, comprising:
a blood pressure bandage for measuring blood pressure;
and the integrated sensor according to claim 4;
wherein the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

12. A health monitor, comprising:
a blood pressure bandage for measuring blood pressure;
and the integrated sensor according to claim 5;
wherein the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

13. A health monitor, comprising:
a blood pressure bandage for measuring blood pressure;
and the integrated sensor according to claim 6;
wherein the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

14. A health monitor, comprising:
a blood pressure bandage for measuring blood pressure;
and the integrated sensor according to claim 7;
wherein the integrated sensor is disposed on a side of the blood pressure bandage in a penetrating mode, the first electrocardio-electrode faces towards the outside of the blood pressure bandage, and the second electrocardio-electrode faces towards the inside of the blood pressure bandage.

15. The health monitor according to claim 8, further comprising a host, wherein the host is connected to the integrated sensor by means of a wire, and the host is connected to the blood pressure bandage by means of the wire and an air tube.

16. The health monitor according to claim 15, wherein the host is provided with a switch and a display screen, and information such as blood pressure, electrocardiogram, heart rate and oxygen saturation is displayed on the display screen.
